(19) United States
(12) Patent Application Publication
(10) Pub. No.: US 2020/0121263 A1
(43) Pub. Date: Apr. 23, 2020

(54) MONITORING A PATIENT IN A MAGNETIC RESONANCE SYSTEM

(71) Applicant: Siemens Healthcare GmbH, Erlangen (DE)

(72) Inventors: Flavio Carinci, Wuerenlingen (CH); Dominik Paul, Bubenreuth (DE); Mario Zeller, Erlangen (DE)

(73) Assignee: Siemens Healthcare GmbH, Erlangen (DE)

(21) Appl. No.: 16/658,516

(22) Filed: Oct. 21, 2019

(30) Foreign Application Priority Data
Oct. 22, 2018 (DE) 102018218057.1

(51) Int. Cl. A61B 5/00; A61B 5/055; A61B 5/11; A61B 90/00; G01R 33/28

(52) U.S. Cl. CPC A61B 5/7292; A61B 5/0077; A61B 5/055; G01R 33/283; A61B 5/1114; A61B 5/1126; A61B 90/39; A61B 5/0035

(57) ABSTRACT

Monitoring a patient in a magnetic resonance system, wherein images of the patient are acquired by a camera in real time, a movement of the patient and a degree of this movement of the patient is determined by evaluating the images, and a symbol symbolizing a part of the patient is depicted, wherein a depicted property of the symbol depicts the degree of movement.

### BACKGROUND

The present disclosure relates to the monitoring of a patient while Magnetic Resonance (MR) data is acquired therefrom by a magnetic resonance system.

The movement of a patient from whom MR data is acquired by means of a magnetic resonance system is one of the most common causes of insufficient image quality or image artifacts. Under some circumstances, movement of the patient can necessitate the repetition of certain recordings resulting in increased time expenditure and hence increased costs in the clinical routine and to a disruption of the daily planning and procedure.

### SUMMARY

It is an object of the present disclosure to improve monitoring of a patient during and/or before acquisition of MR data by means of a magnetic resonance system.

According to the disclosure, this object is achieved by a method for monitoring a patient in a magnetic resonance system, by a magnetic resonance system, by a computer program product, and by an electronically readable data carrier.

In the context of the present disclosure, a method is provided for monitoring a patient in a magnetic resonance system.

The method comprises acquiring images of the patient by a camera in real time. In this step, the patient is so-to-speak monitored with the aid of the camera in that in particular images of the patient are recorded continuously with the camera. Herein, the camera is in particular directed at the part of the patient from which MR data is to be acquired.

The method also comprises determining a movement of the patient and a degree of the movement of the patient by evaluating the images. In this step, the evaluation of the images on the one hand determines or detects the movement of the patient. Herein, in particular the movement is determined of the part of the patient from which the MR data is to be acquired. In addition, the evaluation of the images determines the degree (i.e. severity or speed) of the movement of the patient.

The method also comprises depicting a symbol symbolizing a part of the patient, wherein a depicted property of the symbol depicts the degree of the movement. In this step, the previously determined movement of the patient and in particular the degree of this movement with respect to the quality of the MR data to be acquired is depicted with the aid of the symbol. Herein, the symbol in particular symbolizes the part of the patient from which the MR data is to be acquired.

The fact that the movement of the patient is detected by the camera (in particular an in-bore camera, i.e., a camera located inside the tunnel of the magnetic resonance system camera) and the degrees of this movement in particular in respect of the acquired MR data or MR data to be acquired (and hence ultimately the image quality of the MR images reconstructed using the MR data) are depicted with the symbol means that the present disclosure offers an operator of the magnetic resonance system the advantage of detecting this degree of movement very quickly and in a self-explanatory manner.

According to a preferred embodiment of the disclosure, the symbol is a head symbol of the head of the patient and/or the property is a color of this symbol.

For example, with this embodiment, a green head symbol can indicate to the operator that movement of the patient detected by means of the camera ensures sufficiently good quality of the MR data to be acquired. On the other hand, a red head symbol can indicate to the operator that the movement of the patient detected by means of the camera is impairing the quality of the MR data to be acquired.

The determination of the movement of the patient can comprise the determination of a direction of the movement of the patient. The depiction of the symbol can comprise the depiction of a movement of the symbol, wherein the direction of the movement of the symbol corresponds to the direction of the movement of the patient.

The fact that the determination of the movement of the patient comprises the determination of the direction of this movement and that this direction is depicted by means of the corresponding movement of the symbol advantageously results in a simple-to-understand depiction of the direction of the movement of the patient. If, for example, the patient turns the head to the right, the three-dimensional depiction of the symbol can, for example, also turn to the right. This enables the operator or the user to estimate the movement of the patient and possibly to issue corresponding instructions or other measures can be taken to ensure the quality of the MR data to be acquired despite the movement of the patient.

Herein, the depiction of the movement by means of the symbol can be depicted by a corresponding movement of the three-dimensional or perspective depiction of this symbol. However, it is also possible to depict the direction of the movement by means of a direction symbol (for example an arrow symbol). For example, an arrow symbol can be superimposed on the head symbol in order to indicate the direction of the movement.

According to one embodiment of the disclosure, the determination and depiction of the degree of the movement enables the following distinctions based on the property of the symbol.

One distinction is the movement of the patient is completely correctable during a measurement with the magnetic resonance system. Correspondingly slow movements of the patient can still be corrected by a measuring system or the magnetic resonance system. A slow movement of this kind could, for example, be depicted to the operator by means of a green-colored symbol.

Another distinction is the movement of the patient is only partially correctable during a measurement with the magnetic resonance system. If the degree or the speed of the movement exceeds a predetermined movement threshold, the disruption caused by the movement of the patient can no longer be completely corrected. An excessively fast movement of this kind could, for example, be depicted to the operator by means of a red-colored symbol.

Another distinction is the movement of the patient is not correctable in any way during a measurement with the magnetic resonance system. Certain movements of the patient or certain situations cannot be corrected. If, for example, a marker used to track movement of the patient moves out of the field of view of the camera, the movement of the patient cannot be detected and hence also cannot be corrected. A case of this kind, can, for example, be depicted to the operator by means of a flashing red symbol or an exclamation mark over a red-colored symbol.

The evaluation of the images can comprise the detection of a marker on the patient. In this case, the depiction of the symbol comprises the fact that the symbol depicts whether the marker is detected or whether the marker is not detected.

As already indicated above, the movement of the patient can be detected with the aid of a marker. For this, such a marker is arranged on the part of the patient from which MR data is to be acquired. The movement of the marker detected with the aid of the images acquired by the camera then corresponds to the movement of the patient to be determined. If the marker (for whatever reason) is not (no longer) detected by the camera, it is no longer possible for movement to be detected by means of the images of the patient acquired by the camera. In such a case, it is advantageous for the operator to intervene in the examination and ensure that the marker is again detected by the camera. It is, therefore, important for the information that the marker is not being detected by the camera to be indicated by means of the symbol (for example, by a flashing red head symbol or by an exclamation mark above the head symbol).

According to a further embodiment of the disclosure, the evaluation of the images comprises a determination whether the camera is calibrated or whether the camera is not calibrated. With this embodiment, the depiction of the symbol depicted by means of the symbol comprises whether the camera is calibrated or whether the camera is not calibrated.

With a camera that is not calibrated, the movement of the patient can at the most be detected insufficiently. It is, therefore, important for the information that the camera is not calibrated to be indicated to the operator by means of the symbol (for example by a flashing red head symbol or by an exclamation mark over the head symbol). Advantageously, the depicted property of the symbol for indicating a non-calibrated camera differs from the depicted property of the symbol for indicating that the marker is not being detected by the camera.

According to a further embodiment of the disclosure, the determination of the movement of the patient and the degree of the movement of the patient comprises the following steps:
determining a three-dimensional vector connecting a point of the patient in one of the images acquired at a first time point with the same point of the patient in one of the images acquired at a second time point; and
determining the degree of the movement in that the length of the vector is divided by a time period elapsing between the first time point and the second time point.

The fact that the images acquired by the camera are used to track a certain point of the patient (for example a point of the marker attached to the patient) over time enables the movement of the patient to be detected. For this, a vector is constructed in three-dimensional space, which starts in a first one of the images at this point and ends in a later second one of the images at the same point. The quotient of the amount of this vector and the time period that elapses between the acquisition of the first image and the acquisition of the second image enables the degree of the movement to be determined so-to-speak as the speed with which the point is moving in space.

According to a further embodiment of the disclosure, the determination of the movement of the patient and the degree of the movement of the patient comprises the following steps:
determining a three-dimensional angle, which is spanned by a point of the patient in one of the images acquired at a first time point, by the same point of the patient in one of the images acquired at a second time point and a corresponding angle vertex; and
determining the degree of the movement in that the angular width of the angle is divided by a time period elapsing between the first time point and the second time point.

Similarly to the case with the above-described embodiments, with this embodiment, the movement of the patient is tracked in that a certain point of the patient (for example a point of a marker attached to the patient) is tracked by means of the images. While in the previous embodiment, the degree is determined based on the length of a vector, with this embodiment, the degree of the movement is determined based on an angle in three-dimensional space. For this, the angular width of this angle is determined, which is itself spanned by the angle vertex, the point of the patient in a first one of the images and the same point in a later second one of the images. The quotient of this angular width and the time period that elapses between the acquisition of the first image and the acquisition of the second image can be used to determine the degree of the movement so-to-speak as the angular velocity of the movement of the point.

According to a further embodiment of the disclosure, a latency elapsing between the acquisition of one of the images and the correction of a movement of the patient detected by means of this image is determined. According to this embodiment, the movement of the patient is classed as completely correctable if a quotient of the length of the vector or the angular width and the latency is below a predetermined threshold value.

Accordingly, the latency defines the time period between the occurrence of the movement of the patient and the correction of the movement by the magnetic resonance system during the acquisition of the MR data from a volume section of the patient. Depending upon the latency, which can, for example be 200 ms, 100 ms or even only 50 ms, the degree of the movement must not be too great in order to ensure that the quality of the acquired MR data is not adversely affected by the movement. Therefore, in the event of a movement of the patient in a certain direction, the quotient of the length of the previously described vector and the latency should be below a predetermined length threshold value in order to ensure that the quality of the acquired MR data, and hence the image quality, is not adversely affected by the movement. Similarly, in the event of a rotational movement of the patient, the quotient of the angular width and the latency should be below a predetermined angle-threshold value to ensure that the quality of the acquired MR data is not adversely affected by the movement.

According to a further embodiment of the disclosure, the determination of the movement of the patient and the degree of the movement of the patient comprises the following steps.

One step is acquiring a plurality of consecutive movements of the patient. In this step, the images of the camera are used to detect at least two movements of the patient that are in direct succession. This means there is no further movement of the patient between these at least two movements.

Another step is determining for each of these movements in each case a degree of the respective one of the movements by evaluating the images. The determination of the degree of the respective movement can be performed in the same way as that described above for a single movement of the patient.

Another step is determining the degree of a total movement that comprises the detected consecutive movements of the patient as a function of the degrees of these movements and a number of these movements. The degree of the total movement is determined, on the one hand, as a function of the degrees of the respective movements that are components of the total movement and, on the other, as a function of the number of these movements. Herein, account is taken, for example, of the fact that a temporally short (i.e. temporally limited) but vigorous movement within a total movement has less influence on the quality of the acquired MR data than a lengthy movement within a total movement.

Herein, the depiction of the symbol comprises the fact that the symbol is depicted in that the property of the symbol depicts the degree of the total movement. The manner in which the property of the symbol depicts the degree of the total movement corresponds to the manner in which the property of the symbol depicts the degree of a movement as described above.

Advantageously, the steps of the acquisition of the images of the patient, the determination of the movement of the patient and the degree of the movement of the patient and the depiction of the symbol can be performed before an actual measurement by means of the magnetic resonance system. An acquisition strategy with which MR data of the patient is acquired by means of the magnetic resonance system can then be determined as a function of the determined degree of the movement of the patient.

If the degree of the movement of the patient is already known before the actual measurement by a corresponding depiction of the symbol according to the disclosure, it is possible to employ measurements or examination strategies that are more suitable for reducing, or entirely avoiding, certain motion artifacts. For example, a Turboflash sequence with MPRAGE contrast (“Magnitization Prepared RApid Gradient Echo” contrast) can be adapted to the depicted degrees of the movement of the patient so that the degree of the movement is not expected to influence the image quality.

The symbol depicted on the display can indicate to the operator of the magnetic resonance system that a movement of the patient detected according to the disclosure was too quick and cannot, therefore, be completely corrected. In addition, according to the disclosure, it is possible to identify that for example, the viewing range of the camera is reduced on account of the coil elements attached to the upper side of the head coil and the tiltability of the head coil such that a marker attached to the patient is no longer detected by the camera. It is also possible for this circumstance to be communicated simply and quickly to an operator of the magnetic resonance system by means of the symbol depicted according to the disclosure.

In particular, the depiction of the symbol on the display enables circumstances (for example movements of the patient) that adversely affect the image quality to be communicated to the operator of the magnetic resonance system in a very simple manner. The operator then has the option of issuing corresponding instructions to the patient, for example via an intercom system of the magnetic resonance system, in order at least to restrict the degree of the movements. For this, in the case of slow movements of the patient that can be detected by the camera and corrected by the magnetic resonance system, the symbol can be adapted in accordance with the movements of the patient. The color (for example green) of the symbol can indicate to the operator that the image quality is not yet being adversely affected. On the other hand, in the case of movements of the patient that are too quick, which can still be detected by the camera but, on account of the latency, can no longer be adequately corrected, the symbol can, for example, be changed to the color red. This indicates to the operator that the operator should communicate with the patient accordingly.

Also provided in the context of the present disclosure is a magnetic resonance system for monitoring a patient that comprises an RF control unit, a gradient control unit, an image sequence controller, a computing unit, a camera and a display. The magnetic resonance system is embodied to use the camera to acquire images of the patient located in the magnetic resonance system in real time, to use the computing unit to determine a movement of the patient and a degree of the movement of the patient by evaluating the images and to depict a symbol symbolizing a part of the patient. Herein, a depicted property of the symbol depicts the degree of the movement.

The advantages of the magnetic resonance system according to the disclosure substantially correspond to the advantages of the method according to the disclosure, which were described above in detail and so will not be repeated here.

The present disclosure also describes a computer program product, in particular software, which can be loaded into a memory of a programmable control facility or a computing unit of a magnetic resonance system. This computer program product can execute all or some of the above-described embodiments of the method according to the disclosure when the computer program product is running in the control facility. Herein, the computer program product may require program means, for example libraries and auxiliary functions in order to implement the corresponding embodiments of the method. In other words, the claim directed at the computer program product should in particular place under protection software with which one of the above-described embodiments of the method according to the disclosure can be executed or which executes this embodiment. Herein, the software can be a source code (for example C++), which still has to be compiled and linked or only has to be interpreted or an executable software code that only needs to be loaded into the corresponding computing unit or control facility for execution.

Finally, the present disclosure discloses an electronically readable data carrier, for example a DVD, a magnetic tape, a hard disk or a USB stick on which electronically readable control information, in particular software (see above), is stored. When this control information (software) is read from the data carrier and stored in a control facility or computing unit of a magnetic resonance system, all the embodiments according to the disclosure of the above-described method can be carried out.

With the present disclosure, images acquired from a patient by means of a camera can be used to detect a movement of the patient. A corresponding evaluation of these images also enables the degree of the movement of the patient to be determined and displayed and in the form of a symbol symbolizing a part of the patient. This advantageously enables the degree of this movement and the influence of this movement on the image quality or the quality of the acquired MR data to be depicted in a manner that is very simple and easy to acquire for an operator of the magnetic resonance system.

### BRIEF DESCRIPTION OF THE FIGURES

The present disclosure will now be described in detail with reference to inventive embodiments and with reference to the figures.

FIG. 1 depicts a magnetic resonance system according to the disclosure.

FIG. 2 depicts a camera attached inside a magnetic resonance system.

FIG. 3 depicts a marker attached to a patient.

FIG. 4 is a flow diagram of the procedure according to the disclosure for monitoring a patient.

### DETAILED DESCRIPTION

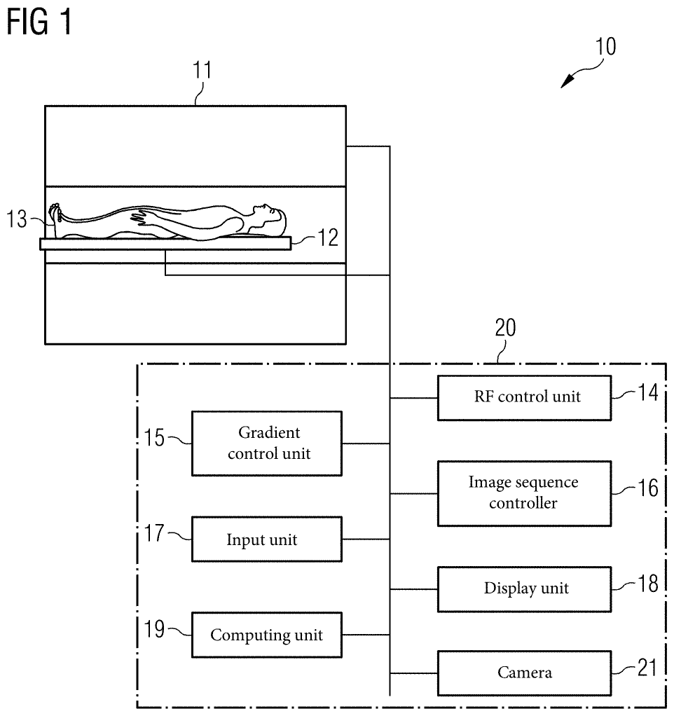

FIG. 1 depicts a magnetic resonance system 10 with which, as explained below, a patient can be monitored according to the disclosure. The magnetic resonance system 10 has a magnet 11 for generating a polarization field BO, wherein an examination subject 13 arranged on a bench 12 is moved into the magnet 11 for the recording of spatially encoded magnetic resonance signals or MR data from the examination subject or the patient 13. The coils used for signal recording, such as a whole-body coil or local coils, are not shown for reasons for clarity. The radiation of RF pulses and switching of magnetic field gradients causes the magnetization generated by the polarization field BO to be deflected out of the equilibrium position and spatially encoded and the resultant magnetization is detected by the receiving coils. The way in which MR images can be generated by radiating RF pulses and switching magnetic field gradients in different combinations and sequences is in principle known to the person skilled in the art and will not be explained in further detail here.

The magnetic resonance system 10 further has a control unit 20 that may be used to control the magnetic resonance system 10. The controller 20 has a gradient control unit 15 for controlling and switching the necessary magnetic field gradients. An RF control unit 14 is provided to control and generate the RF pulses for deflecting the magnetization. An image sequence controller 16 controls the sequence of magnetic field gradients and RF pulses and hence indirectly the gradient control unit 15 and the RF control unit 14. An operator can control the magnetic resonance system 10 via an input unit 17 and MR images and other information required for control purposes can be displayed on a display unit 18. A computing unit 19 with at least one processor unit (not shown) is provided to control the different units in the control unit 20 and to carry out computing operations. Furthermore, a camera 21 is provided with which images of the patient 13 can be acquired. The computing unit 19 is embodied to calculate the MR images from the acquired MR signals and to use the acquired images to determine a movement of the patient 13 and a degree of the movement. A symbol (for example a head symbol) is then used to depict the degree of the movement on the display 18.

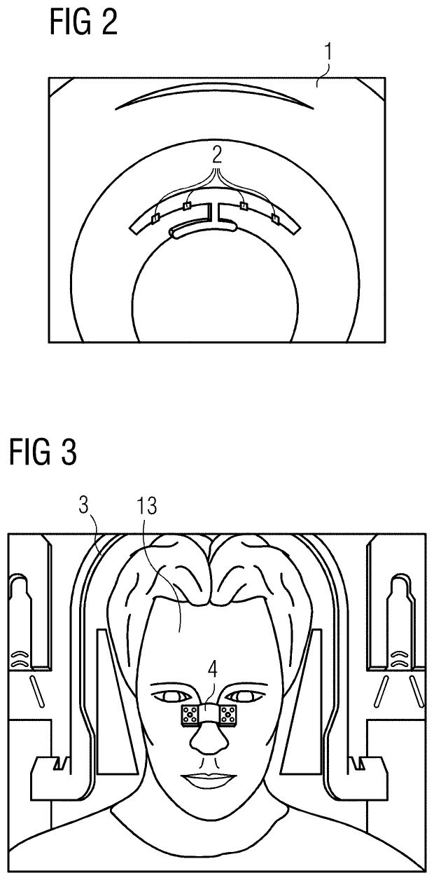

FIG. 2 depicts an in-bore camera arranged on the upper side of the tunnel 1 of the magnetic resonance system 10. This camera acquires the images of the patient by means of four slots or camera windows 2 arranged on the upper side of the tunnel 1 so the camera acquires four different views of the patient 13.

FIG. 3 depicts the head of a patient 13 wearing a head coil 3 located inside the magnetic resonance system 10. To improve tracking of the movement of the head of the patient 13, the patient 13 wears a kinetic marker 4, which is attached to the patient 13 on the bridge of the nose.

Whenever the marker 4 is visible to the camera 21, it is possible to track a movement of the head of the patient 13 using the images acquired from the patient 13 in that, for example, a point of this marker 4 is tracked using the images.

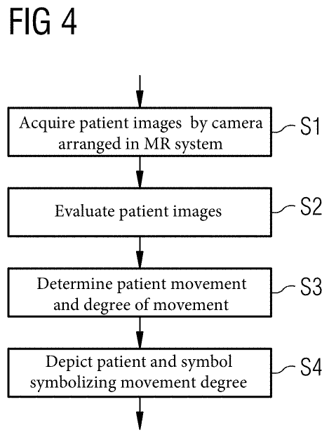

FIG. 4 depicts a flow diagram of a method according to the disclosure for monitoring a patient 13.

In the first step S1, images of the patient 13 are acquired by means of a camera 21 arranged in a magnetic resonance system 10. Herein, the patient 13 is located in the magnetic resonance system 10 to enable this magnetic resonance system 10 to acquire MR data of a certain part (for example the head) of the patient 13.

In a second step S2 following the first step S1, these images are evaluated in order to determine a movement of the patient 13 and a degree of this movement in a third step S3. The degree of this movement is then depicted in a fourth step S4 with the aid of a symbol symbolizing a part (for example the head) of the patient 13. Herein, a depicted property of this symbol is used to depict the degree of the movement on the display 21 such that an operator of the magnetic resonance system is advantageously able to detect very quickly whether the degree of the movement is having an adverse effect on the image quality.

For example, the color of the symbol can change as a function of the movement pattern of the patient 13 determined by means of the images. A green head symbol can, for example, indicate to the operator of the magnetic resonance system 10 that the movement of the patient 13 currently determined by means of the images is not yet having an adverse effect on the image quality. On the other hand, a red head symbol can indicate to the operator that the movement of the patient 13 currently determined is already having an adverse effect on the image quality.

1. A method for monitoring a patient in a magnetic resonance system, comprising:
acquiring images of the patient by a camera in real time;
determining a movement of the patient and a degree of the movement of the patient by evaluating the images; and
depicting a symbol symbolizing a part of the patient, wherein a depicted property of the symbol depicts the degree of the movement.

2. The method as claimed in claim 1, wherein the symbol is a head symbol of the head of the patient or the property is a color of the symbol.

3. The method as claimed in claim 1,
wherein the determination of the movement of the patient comprises the determination of a direction of the movement of the patient, and
wherein the depiction of the symbol comprises a depiction of a movement of the symbol, wherein a direction of the movement of the symbol corresponds to a direction of the movement of the patient.

4. The method as claimed in claim 1, wherein the determination and depiction of the degree enables a distinction based on the property of the symbol as follows:
the movement of the patient is completely correctable during a measurement with the magnetic resonance system,
the movement of the patient is only partially correctable during a measurement with the magnetic resonance system, or
the movement of the patient is not correctable in any way during a measurement with the magnetic resonance system.

5. The method as claimed in claim 1,
wherein the evaluation of the images comprises the detection of a marker on the patient, and
wherein the depiction of the symbol depicts whether the marker is detected or whether the marker is not detected.

6. The method as claimed in claim 1,
wherein the evaluation of the images comprises a determination whether the camera is calibrated or whether the camera is not calibrated, and
wherein the depiction of the symbol comprises that the symbol depicts whether the camera is calibrated or whether the camera is not calibrated.

7. The method as claimed in claim 1, wherein the determination of the movement of the patient and the degree of the movement of the patient comprises:
determining a three-dimensional vector connecting a point of the patient in one of the images acquired at a first time point with the same point of the patient in one of the images acquired at a second time point; and
determining the degree of the movement in that the length of the vector is divided by a time period elapsing between the first time point and the second time point.

8. The method as claimed in claim 1, wherein the determination of the movement of the patient and the degree of the movement of the patient comprises:
determining a three-dimensional angle, which is spanned by a point of the patient in at least one of the images acquired at a first time point and the same at least one point of the patient in one of the images, acquired at a second time point and a corresponding angle vertex, and
determination of the degree of the movement in that a angular width of the three-dimensional angle is divided by a time period elapsing between the first time point and the second time point.

9. The method as claimed in claim 7,
wherein a latency elapsing between the acquisition of one of the images and a correction of a movement of the patient detected by means of this image, and
wherein the movement of the patient is classed as completely correctable if a quotient of the length of the vector and the latency is below a predetermined threshold value.

10. The method as claimed in claim 7,
wherein a latency elapsing between the acquisition of one of the images and a correction of a movement of the patient detected by means of this image, and
wherein the movement of the patient is classed as completely correctable if a quotient of the angular width and the latency is below a predetermined threshold value.

11. The method as claimed in claim 1,
wherein the determination of the movement of the patient and the degree of the movement of the patient comprises:
acquiring a plurality of consecutive movements of the patient,
determining for each of these movements in each case a degree of the respective one of the movements by evaluating the images, and
determining the degree of a total movement that comprises the detected consecutive movements of the patient as a function of the degrees of these movements and a number of these movements, and
wherein the depiction of the symbol comprises:
depicting the symbol in that the property of the symbol depicts the degree of the total movement.

12. The method as claimed in claim 1,
wherein the steps of the acquisition of the images of the patient, the determination of the movement of the patient, and the degree of the movement of the patient, and the depiction of the symbol, are performed before an actual measurement by means of the magnetic resonance system, and
wherein an acquisition strategy with which magnetic resonance data of the patient are acquired by means of the magnetic resonance system is determined as a function of the determined degree of the movement of the patient.

13. A magnetic resonance system for monitoring a patient, wherein the magnetic resonance system comprises a Radio Frequency (RF) controller, a gradient controller, an image sequence controller, a computer, a camera, and a display, wherein the magnetic resonance system is configured to:
use the camera to acquire images of the patient located in the magnetic resonance system in real time;
use the computer to determine a movement of the patient and a degree of the movement of the patient by evaluating the images; and
depict a symbol on the display symbolizing a part of the patient, wherein a depicted property of the symbol depicts the degree of the movement.

14. The magnetic resonance system as claimed in claim 13, wherein the magnetic resonance system is configured to carry out the method as claimed in claim 1.

15. A non-transitory computer program product that comprises a program and loadable directly into a memory of a programmable control facility of a magnetic resonance system with computer configured to execute the steps of the method as claimed in claim 1 when the program is executed in the control facility of the magnetic resonance system.
